(19) United States
(12) Patent Application Publication
(10) Pub. No.: US 2023/0400705 A1
(43) Pub. Date: Dec. 14, 2023

(54) FLEXIBLE MASK WITH CORRECTIVE LENSES

(71) Applicant: Philip E. Klein, East Meadow, NY (US)

(72) Inventor: Philip E. Klein, East Meadow, NY (US)

(21) Appl. No.: 18/209,684

(22) Filed: Jun. 14, 2023

Related U.S. Application Data
(60) Provisional application No. 63/352,066, filed on Jun. 14, 2022.

(51) Int. Cl. G02C 5/00; G02C 3/00

(52) U.S. Cl. CPC G02C 5/006; G02C 3/003

(57) ABSTRACT

A flexible mask includes a body portion and a head strap portion to hold the body portion over a user's eyes. The body portion includes lenses aligned with the user's eyes to aid the user's vision and includes cover elements that are movable to cover and uncover the lenses as desired.

The present application claims benefit of and priority to U.S. Provisional Patent Application Ser. No. 63/352,066 entitled FLEXIBLE MASK WITH CORRECTIVE LENSES filed Jun. 14, 2022, the entire content of which is incorporated by reference herein.

### BACKGROUND

### Field of the Disclosure

The present invention relates to a flexible mask including at least one corrective lens or optical film sufficient to aid a user's vision.

### Related Art

A large proportion of the American public uses corrective lenses to aid their vision. Indeed, about 30% of adults alone use lenses to aid in reading. All of these users, however, have to deal with a common problem, damage to their glasses.

Conventional corrective lenses, such as reading glasses, or other glasses, typically include rigid frames to support the lenses in a desired position relative to the user's eyes. While such frames are effective for supporting the lenses, the rigid nature of the frames tends to make them uncomfortable if they are worn for a long period of time. In addition, while the rigid frames are typically durable, they are subject to bending and deforming, which may make them unsuitable for use.

Bending and deformation may occur when a user drops their glasses and increasingly is the result when the user forgets to take their glasses off while engaging in certain activities such as sports or even sleeping. Indeed, about 89% of adults use electronic devices such as smart phones or pads to view content or read prior to going to sleep. About 60% of adults report falling asleep while reading, which can result in damage to the user's glasses.

Accordingly, it would be beneficial to provide a flexible mask with corrective lenses that avoids these and other problems.

### SUMMARY

It is an object of the present disclosure to provide a flexible mask that includes corrective lenses.

A mask in accordance with an embodiment of the present disclosure includes: a flexible body; a strap connected to the flexible body and configured to secure the flexible body to a user's face; at least one lens mounted in the flexible body such that the at least one lens is aligned with an eye of the user when the flexible body is secured to the user's face; and at least one cover element movably mounted on the flexible body to move between a closed position in which the at least one cover element covers the at least one lens and an open position in which the at least one lens is uncovered such that the user sees through the at least one lens.

In embodiments, the flexible body includes: a first opening configured to receive the at least one lens; and a first fastening element configured to hold the at least one cover element over the first opening to cover the at least one lens.

In embodiments, the flexible body includes: an outer surface made of a soft material; and at least one foam element covered by the soft material.

In embodiments, the soft material is silk.

In embodiments, the soft material is satin.

In embodiments, the foam is memory foam.

In embodiments, the first fastening element is a hook and loop type fastener.

In embodiments, the mask includes: a second lens mounted in the flexible body such that the second lens is aligned with a second eye of the user when the flexible body is secured to the user's face; and a second cover element movably mounted on the flexible body to move between a closed position in which the second cover element covers the second lens and an open position in which the second lens is uncovered such that the user sees through the second lens.

In embodiments, the flexible body further includes: a second opening configured to receive the second lens; and a second fastening element configured to hold the second cover element over the second opening to cover the second lens.

In embodiments, the strap is integrated into the flexible body.

In embodiments, a securing element is configured to adjustably secure the strap to the flexible body such that a length of the strap is adjustable.

In embodiments, a securing element is configured to releasably secure the strap to the flexible body.

In embodiments, the flexible body comprises a first bezel element surrounding the first opening and configured to receive the at least one lens.

In embodiments, the first bezel element includes a slot configured to receive the at least one lens.

In embodiments, the at least one lens includes a peripheral seal extending around a periphery of the at least one lens and sized to fit in the slot.

In embodiments, the at least one lens is a corrective lens.

In embodiments, the flexible body is configured such that the at least one lens is mounted in the flexible body at a desired pantoscopic tilt.

In embodiments, the desired pantoscopic tilt is between 5 degrees and 15 degrees.

In embodiments, the at least one lens is a blue light blocking lens.

In embodiments, the at least one lens is mounted in the flexible body to provide a desired vertex length between a cornea of the user and a back of the at least one lens.

In embodiments, the body is configured to provide a desired pupillary distance between a center of the at least one lens and a center of the second lens.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1A illustrates a front view of a flexible mask with corrective lenses in which the corrective lenses are covered in accordance with an embodiment of the present disclosure;

FIG. 1B illustrates the front view of the flexible mask of FIG. 1A with the corrective lenses uncovered in accordance with an embodiment of the present disclosure;

FIG. 2A illustrates a front view of a flexible mask with corrective lenses in which the corrective lenses are covered in accordance with another embodiment of the present disclosure;

FIG. 2B illustrates the front view of the flexible mask of FIG. 2A with the corrective lenses uncovered in accordance with another embodiment of the present disclosure;

FIG. 2C illustrates a front view of a flexible mask with corrective lenses with the lenses uncovered in accordance with another embodiment of the present disclosure;

FIG. 2D illustrates the front view of the flexible mask of FIG. 2C with the lenses covered in accordance with another embodiment of the present disclosure;

FIG. 2E illustrates a rear view of the flexible mask of FIG. 2C in accordance with another embodiment of the present disclosure;

FIG. 3A illustrates an exemplary rear view of a flexible mask with corrective lenses including exemplary dimensions in accordance with another embodiment of the present disclosure;

FIG. 3B illustrates a more detailed view of the nose piece of the flexible mask of FIG. 3A in accordance with another embodiment of the present disclosure;

FIG. 3C illustrates a more detailed view of one of the eye openings of the flexible mask of FIG. 3A in accordance with another embodiment of the present disclosure;

FIG. 4A illustrates an exemplary securing element that may be used in the flexible masks of FIGS. 1A, 2A, 2C and 3A in accordance with another embodiment of the present disclosure; and

FIG. 4B illustrates multiple exemplary securing elements that may be used in the flexible masks of FIGS. 1A, 2A, 2C and 3A in accordance with another embodiment of the present disclosure;

FIG. 5 illustrates a side view of the flexible mask of DIG. 2C illustrating a tilt of a lens provided therein in accordance with an embodiment of the present application;

FIG. 6A illustrates a detailed view of the lens opening 210 in the flexible mask of FIG. 2C;

FIG. 6B illustrates a detailed view of a lens slot formed adjacent to the lens opening of FIG. 6A to receive the lens;

FIG. 7 illustrates a detailed view of a lens suitable for use in the flexible mask of FIG. 2C showing a band around the periphery thereof that may be received in the slot of FIG. 6B; and

FIG. 8 illustrates a detailed view of the lens as well as the layers of material that may be provided to secure the lens in the lens opening in the flexible mask of FIG. 2C.

### DETAILED DESCRIPTION OF THE EXEMPLARY EMBODIMENTS

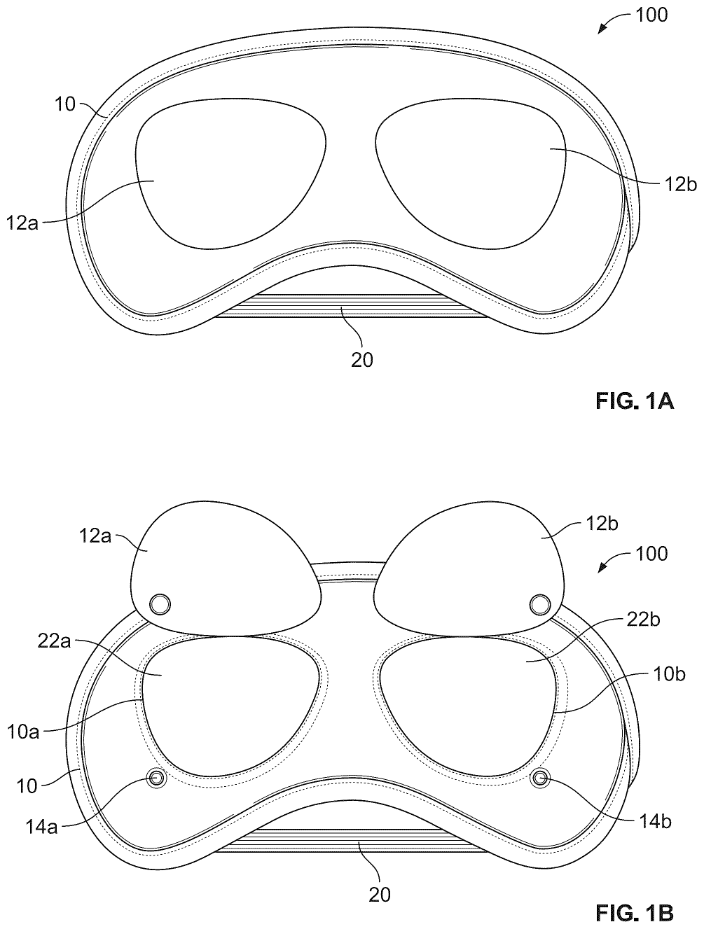

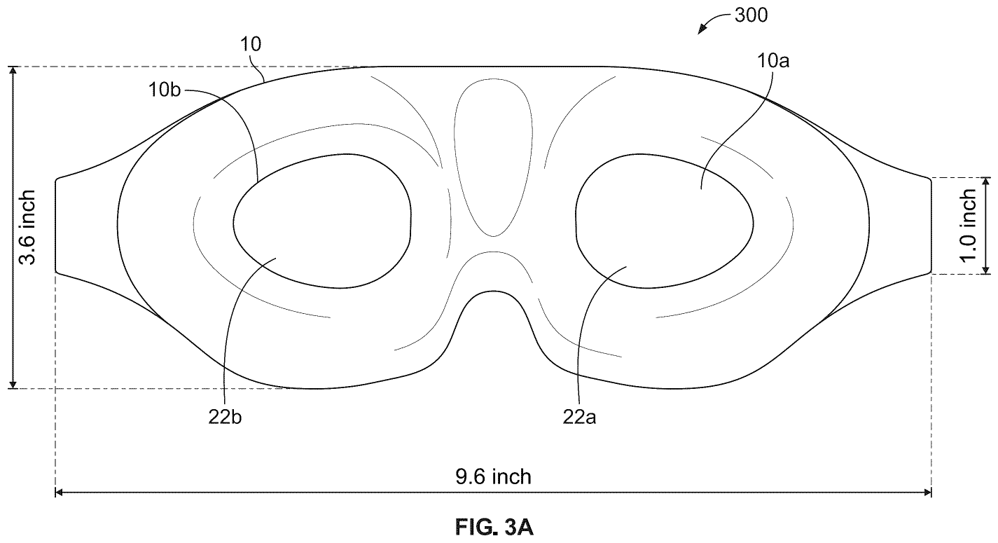

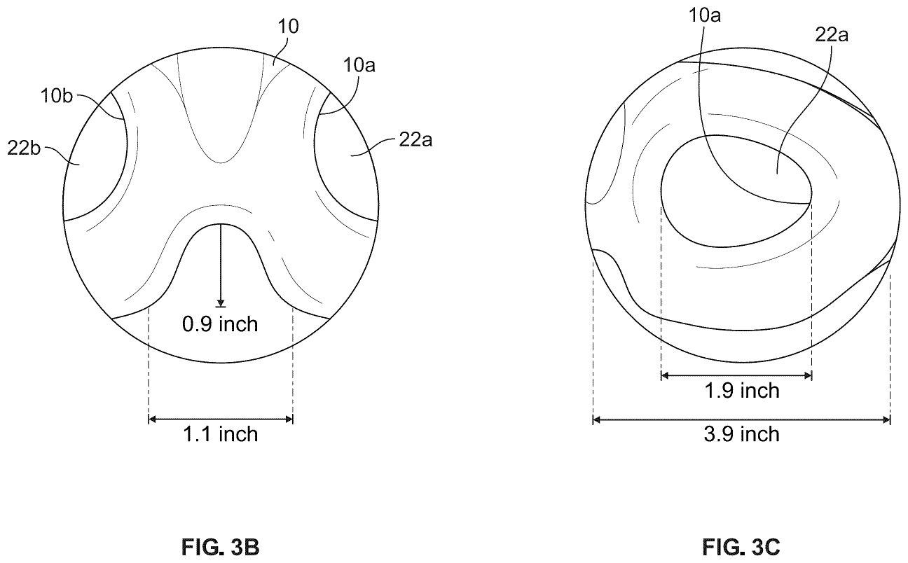

A flexible mask 100 in accordance with an embodiment of the present disclosure is illustrated in FIG. 1A. In embodiments, the flexible mask 100 may include a body portion 10 configured to be positioned over the eyes of a user and a head strap 20 connected thereto and configured to extend around the user's head to hold the body portion 10 in place over the user's eyes. In embodiments, the body portion 10 may include a pair of eye openings 10a, 10b (see FIG. 1B) formed in a front thereof and positioned such that they align with the eyes of the user when the mask 100 in in place over the user's eyes at an angle that that incorporates the desired pantoscopic tilt and at an appropriate vertex distance. In embodiments, lenses 22a, 22b are positioned in the respective openings 10a, 10b. In embodiments, the lenses 22a, 22b may be corrective lenses. In embodiments, the lenses 22a, 22b, may be prescription lenses. In embodiments, the lenses 22a, 22b may be blue light blocking lenses. In embodiments, the openings 10a, 10b may be spaced apart to align with a user's eyes and may be consistent with industry standards. In embodiments, the lenses 22a, 22b may be mounted to provide a pupillary distance, the distance between two pupils, or the centers of the lenses, of about 60 mm to 64 mm. In embodiments, the spacing between the openings 10a, 10b may be larger or smaller depending on the size of the user's head. In embodiments, the mask 100 may come in different sizes, for example small, medium, large, extra-large, etc. to accommodate different users. In embodiments, the mask 100 may also be provided in youth or child sizes. FIG. 3A illustrates exemplary dimensions of a mask 100, 200, 300, 400 in accordance with embodiments of the present disclosure, however, as noted above, the mask may be smaller or larger, if desired.

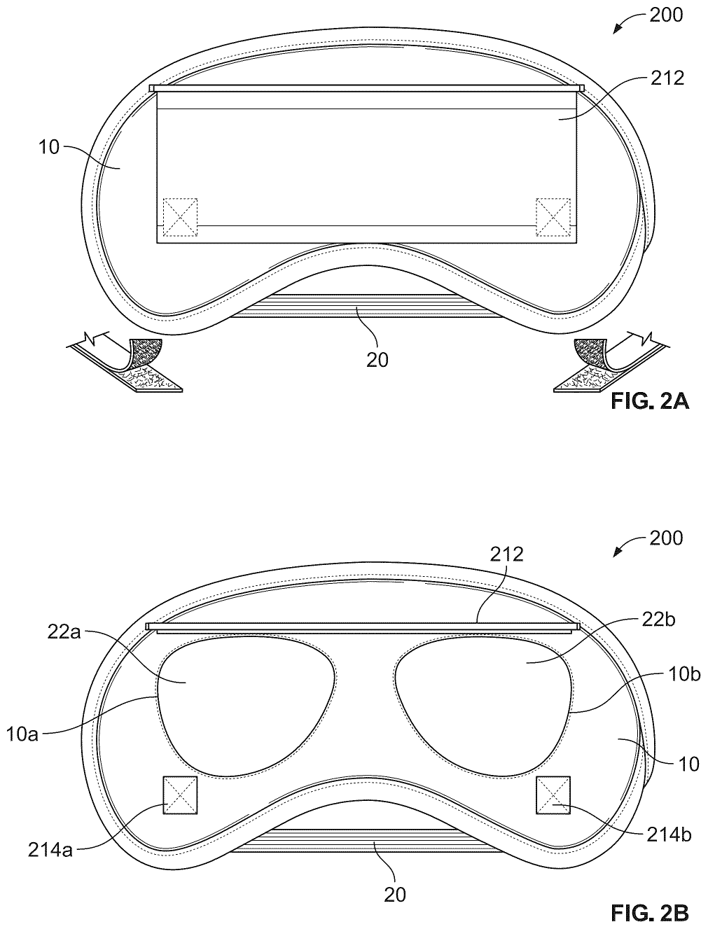

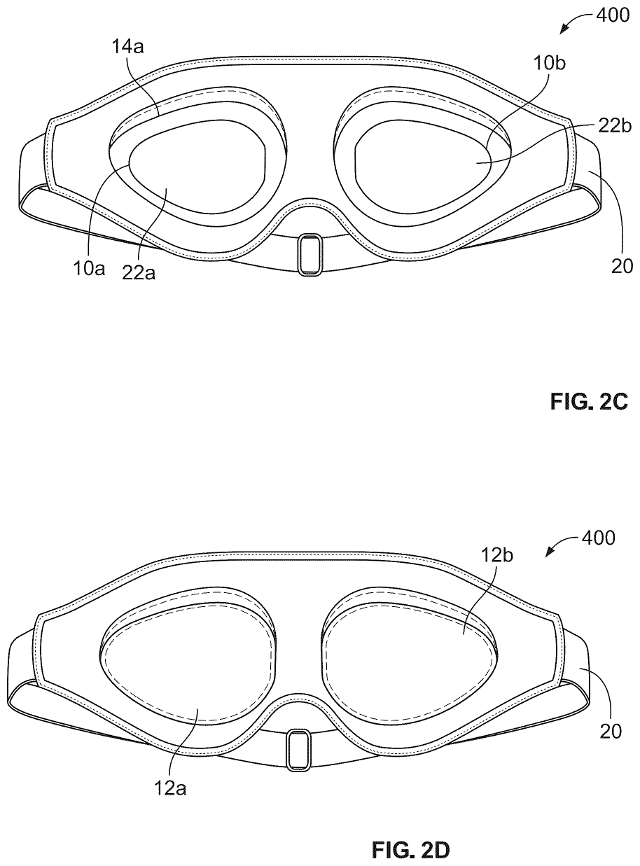

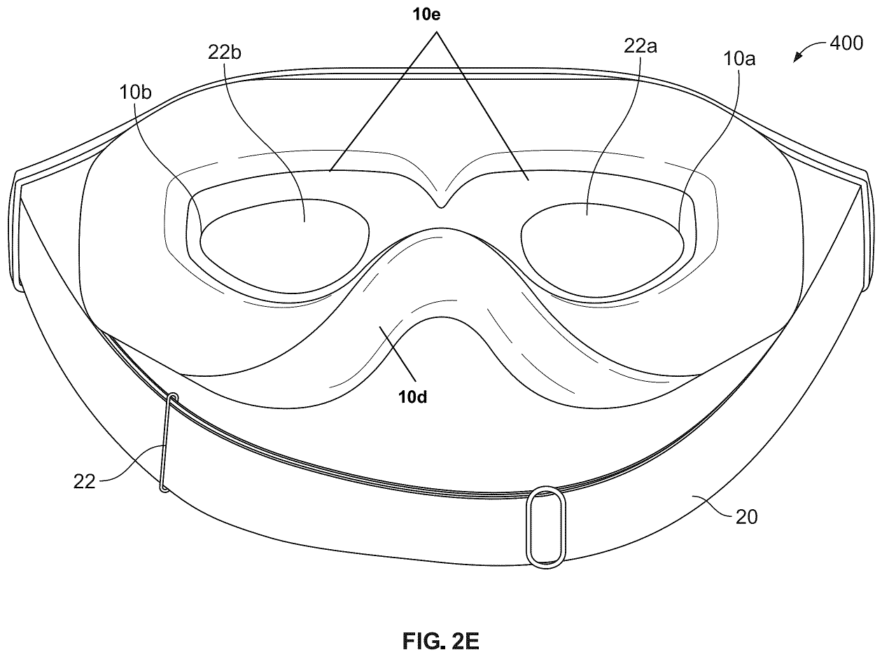

In embodiments, the body 10 may be made of a flexible material, such as fabric. In embodiments, the body 10 may include padding or other filling to provide comfort while in place on the user's head. In embodiments, the body 10 may be or include a high quality, lightweight, soft/comfortable breathable foam (see FIGS. 2E, 3A and 4, for example) so that it is pliable to accommodate faces of various sizes. In embodiments, the flexible body 10 will flex to conform to the user's face while positioning the lenses 22a 22b in front of the user's eyes and spaced appropriately to allow the user to see through the lenses. In embodiments, the body 10 will rest over the user's eyes and provide enough pressure to maintain its facial positioning and align the lenses 22a, 22b to aid vision and focus. In embodiments, the portions of the body 10 around the openings 10a,10b may be contoured so as to place no pressure on the eyes and may be vented to provide breathability. In embodiments, the flexible nature of the body 10 prevents deformation which would prevent future use. In embodiments, the body 10 may have a width to accommodate small, medium, large and extra-large heads and may also be provided in youth or children's sizes, as noted above. In embodiments, the body 10 may be any suitable size such that the user's eyes are completely covered when desired. In embodiments, the body 10 may be larger or smaller to accommodate users with different head sizes.

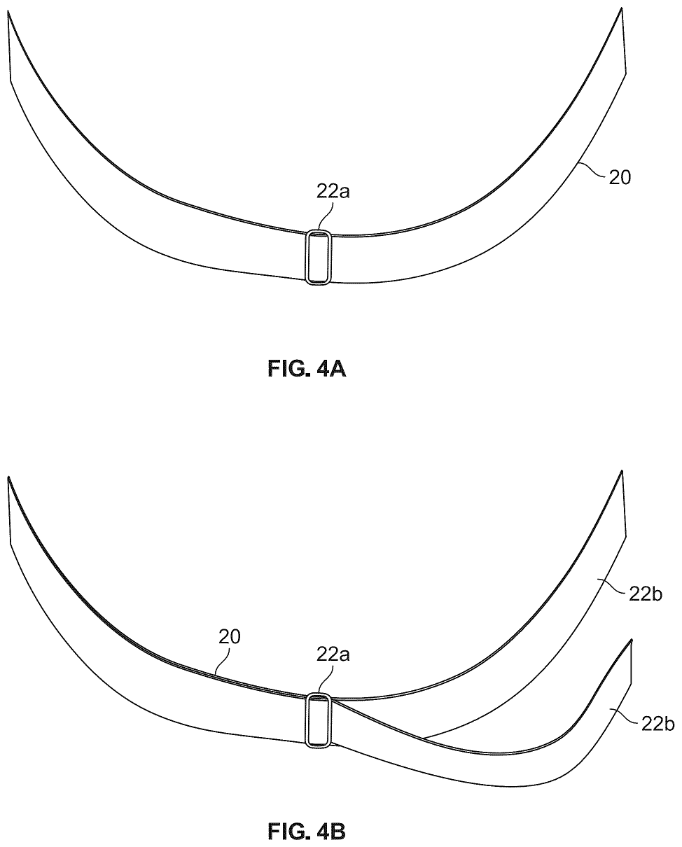

In embodiments, the head strap 20 may be integrated into the body 10. In embodiments, the body 10 may be elongated to wrap around the user's head in which case a separate head strap 20 may not be used. In embodiments, the body 10 may include an elastic portion that may provide for some stretch to fit securely onto the user's head. In embodiments, the strap 20 may include or be made of an elastic material to allow for a secure fit. In embodiments, the body 10 may be elongated to wrap around a user's head and may include a securing element or elements at opposite ends thereof that may be used to join the two ends and secure the body 10 to the user's head. In embodiments, the securing elements may allow for adjustment of the length of the body 10 to accommodate heads of different sizes. In embodiments, the body 10 may include cushioning or foam at the ears to minimize noise. In embodiments, the head strap 20 may be adjustable and may include a securing element 24a (see FIGS. 4A, 4B) to allow for adjustment of the size of the strap 20. In embodiments, multiple securing elements 24a, 24b may be used to allow for both adjustment of length and for securing the strap 20 around the user's head (see FIG. 4B). In embodiments, the securing element 24b may be or include a snap, hook and loop as in FIG. 4B, or plastic mating fastener, to name a few. In embodiments, more than two securing elements may be used. In embodiments, two securing elements may be used, with one provided at each temple of the user, for example. In embodiments, the head strap 20 may be implemented by a pair of ear loops provided at opposite ends of the body 10 and configured to extend behind the user's ear on each side of the user's head. In embodiments, the user may place the body 10 over their eyes and extend the loops back to be secured behind the user's ears to hold the body in place.

In embodiments, the body 10 may include cover elements 12a, 12b that may be moved from a closed position, as shown in FIG. 1A, for example, in which they cover the lenses 22a, 22b and openings 10a, 10b to an open position as illustrated in FIG. 1B where the lenses 22a, 22b are uncovered such that the user can see while wearing the mask 100. In embodiments, when the cover elements 12a, 12b are in the closed position, they shield the user's eyes such that the mask 100 may be used as a sleep mask to block out light and aid the user's sleep. That is, in embodiments, the mask 100 may be used as a sleep mask to aid user sleep. In embodiments, the cover elements 12a, 12b may be secured in the closed position using the fastening elements 14a, 14b, for example. In embodiments, the fastening elements 14a, 14b may be snap fasteners, as illustrated in FIG. 1B, for example, that mate with corresponding snaps provided on the cover element 12a, 12b. In embodiments, the fastening elements 14a, 14b may be hook and loop type fasteners, hook and eye fasteners, or any other suitable fastener. In embodiments, any suitable fastening elements that releasably secure the cover elements 12a, 12b in the closed position may be used.

In embodiments, the cover elements 12a, 12b may be lifted into the open position, as illustrated in FIG. 1B, to allow the user to see while still wearing the mask 100. In embodiments, fasteners may be provided on the body 10 to hold the cover elements 12a, 12b in the open position. In embodiments, the fasteners may be or include a snap, hook and loop type fastener, hook and eye fastener or any other suitable fastener. In embodiments, the cover elements 12a, 12b may be removed from the body 10. In embodiments, the mask 100 may not include the cover elements 12a, 12b such that the lenses 12a, 12b are always uncovered. As noted above, the lenses 22a, 22b may be mounted in the openings 10a, 10b formed in the body 10. In embodiments, the lenses 22a, 22b may be corrective lenses that may help the user to see when reading, working or watching TV or other content. The lenses 22a, 22b may be prescription lenses or blue light blocking lenses. In embodiments, the lenses 22a, 22b may be made of glass, plastic, polycarbonate or any other suitable material. In embodiments, the lenses 22a, 22b may be or include optical film that may be used to aid the user's vision.

In embodiments, the body 10 may include pockets aligned with the openings 10b which may be configured to receive the lenses 22a, 22b and position them in the openings 10a, 10b. In embodiments, the lenses 22a, 22b are removably mounted in the openings 10a, 10b such that they can be switched in and out and different corrective lenses may be used as necessary. In embodiments, a top seam of the pocket, or pockets, may include a closure element such as a snap or zipper to close the top seam after the lens is inserted to hold the lens in place. In embodiments, the top seam may be sewn shut or may be closed using ultrasonic welding or heat treatment. In embodiments, the lenses 22a, 22b may be secured to the body 10 via adhesive. In embodiments, the lenses 22a, 22b may be sewn into place. In embodiments the lenses 22a, 22b may be ultrasonically welded into place. In embodiments, heat sealing may be used to secure the lenses 22a, 22b into place. In embodiments, where optical film is used, the optical film may be affixed to clear lenses. In embodiments, the optical film may be directly secured to the body 10 by sewing, for example.

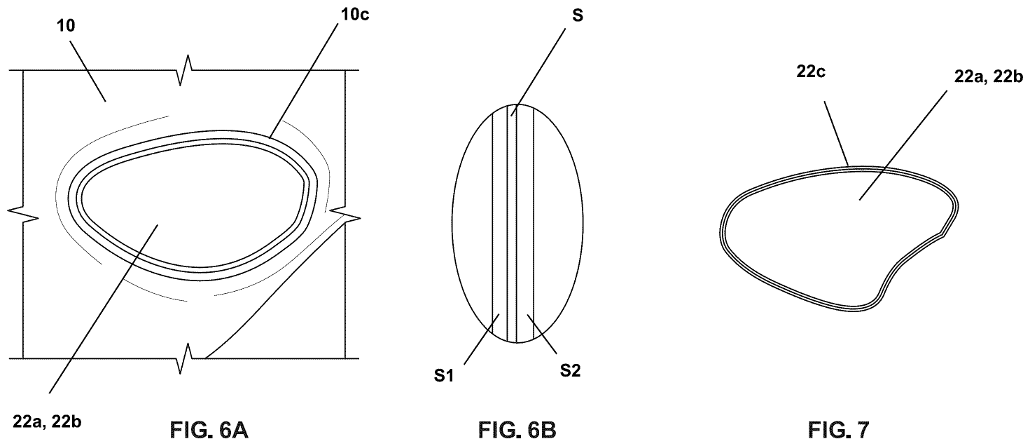

In embodiments, the body 10 may include a bezel 10c (See FIG. 6A, for example) formed around the openings 10a, 10b which may be made of plastic, metal, fabric or any other suitable material and configured to receive the lenses 22a, 22b. In embodiments, the bezel 10c may be configured to removably receive the lenses 22a, 22b. In embodiments, the bezel 10c may be provided on the body 10. In embodiments, the bezel 10c or a portion thereof may be provided on the lenses 22a, 22b. In embodiments, a portion of a bezel structure 10c may be included on the lenses 22a, 22b and another portion may be included on the body 10 as is further discussed below.

In embodiments, the head strap 20 may be an elastic band connected to opposing ends of the body 10 to hold it in place over the user's eyes. In embodiments, the head strap 20 may be attached to opposite ends of the body 10 and may include an adjustable closure (securing elements 24a, 24b, for example) to connect ends of the strap to secure the body 10 in place over the user's eyes as shown in FIG. 4A, 4B, for example. As noted above, in embodiments, the body 10 may integrate the head strap 20 in which case a separate head strap 20 may not be used. In embodiments, the head strap may extend vertically up and over the user's head. In embodiments, the head strap 20 may include a portion that extends vertically up and over the user's head and connects with another portion extending around the user's head. In embodiments, the head strap may be modified to provide the arms used in conventional eyeglasses that hook behind the user's ear. In embodiments, the body 10 may extend around the user's head and beyond the user's ears and may include ear openings to receive the user's ears and hold the mask 100 in place on the user's head.

FIG. 2A illustrates another embodiment of a flexible mask 200. In embodiments, the mask 200 may include the flexible body 10 that may be secured in place over a user's eyes using the head band 20 in a manner similar to that described above. In embodiments, the flexible body 10 may include the openings 10a, 10b formed therein in which the lenses 22a, 22b may be mounted in the manner described above. In embodiments, the mask 200 may include a movable cover element 212 that may be moved from a closed position, as illustrated in FIG. 2A in which both the lenses 22a, 22b are covered to an open position, as illustrated in FIG. 2B, in which the lenses 22a, 22b are uncovered and the user can see without taking off the mask. In embodiments, the cover element 212 may fold upward or otherwise away from the lenses 22a, 22b to allow the user to see. In embodiments, the body 10 may include fastening elements 214a, 214b which may be used to secure the cover element 212 in the closed position, which may be snaps, hook and loop type fasteners, hook and eye type fasteners or any suitable fastening element. In embodiments, one or more fasteners may be used to hold the cover element 212 in the open position as well. In embodiments, such fasteners may be mounted on the body 10 or may be detachable. In embodiments, the cover element 212 may be removed completely from the flexible body 10. In embodiments, the mask 200 may not include the cover element 212 at all such that the lenses 22a, 22b may be exposed at all times. In embodiments, the fastening elements 214a, 214b may be or may be similar to the fastening elements 14a, 14b discussed above.

FIG. 3A illustrates another embodiment of a mask 300 including padding around the openings 10a, 10b and around the nose piece to add comfort. The mask 300 is otherwise similar in construction to the masks 100, 200 discussed above. As noted above, the padding may be foam such as that discussed above.

FIG. 2C illustrates another embodiment of a flexible mask 400. In embodiments, the mask 400 may include the flexible body 10 that may be secured in place over a user's eyes using the head band 20 in a manner similar to that described above. In embodiments, the flexible body 10 may include the openings 10a, 10b formed therein in which the lenses 22a, 22b may be mounted in the manner described above. In embodiments, the mask 400 may include cover elements 12a, 12b that are movable from a closed position (see FIG. 2D) in which both the lenses 22a, 22b are covered, to an open position shown in FIG. 2C, in which the lenses 22a, 22b are uncovered and the user can see without taking off the mask. In embodiments, the cover elements 12a, 12b may fold upward or otherwise away from the lenses 22a, 22b to allow the user to see. In embodiments, the cover elements 12a, 12b may be completely removed from the body 10 to allow the user to see. In embodiments, the body 10 may include fastening elements 14a, 14b which may be used to secure the cover elements 12a, 12b in the closed position, which may be snaps, hook and loop type fasteners, as shown in FIG. 2C, hook and eye type fasteners or any suitable fastening element. In embodiments, one or more fasteners may be used to hold the cover elements 12a, 12b in the open position as well. In embodiments, such fasteners may be mounted on the body 10 or may be detachable. In embodiments, the cover elements 12a, 12b may be removed from the flexible body 10 entirely. In embodiments, the mask 400 may not include the cover elements 12a, 12b at all such that the lenses 22a, 22b may be exposed at all times. In embodiments, the fastening elements 14a, 14b hold the cover elements 12a, 12b in place on the mask 400 in substantially the same manner as discussed above with respect to the mask 100.

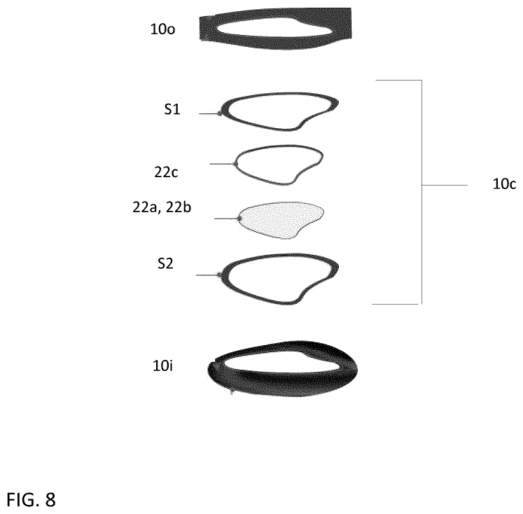

FIG. 2E illustrates a rear view of the mask 400 of FIGS. 2C and 2D. In embodiments, as can be seen in FIG. 2E, the mask 400 may include a memory foam filling to provide a cushioned body facing surface in contact with the wearer's body when the mask 400 is in place. A similar cushioned surface is shown in FIG. 3A. In embodiments, a nose cushion 10d is provided for comfort around the user's nose. In embodiments, the cushion material may be configured to provide deep eye sockets 10e surrounding each of the eye openings 10a, 10b for improved user comfort. In embodiments, the cushioned foam portion may be covered by an outer covering 10o on the front facing portion (see FIG. 8, for example), 10i on the rear or user facing portion, made of a soft and smooth fabric for comfort. In embodiments, the soft outer covering may be silk or satin, as noted above. In embodiments, the foam cushion may be memory foam.

In embodiments, the mask 100, 200, 300, 400 may include one or more spacers which may be mounted in the body 10 between the openings 10a, 10b which may be used to maintain a distance between the openings 10a, 10b and the lenses 22a, 22b mounted therein. In embodiments, the foam or other filling or cushion is sufficiently resilient to maintain the distance between the openings 10a, 10b and the lenses 22a, 22b mounted therein. In embodiments, the spacers may be used to prevent or limit folding of the flexible body 10 between the openings 10a, 10b such that the space between the openings may be maintained. In embodiments, the foam cushion portion provided in the body 10 may provide sufficient rigidity to maintain the spacing between the eye openings 10a, 10b.

In embodiments, the body 10 may include the lenses 22a, 22b sheathed between two swatches of cloth or a synthetic fabric material. In embodiments, the body 10 may include additional pliable structural elements connecting the lenses 22a, 22b and sheathed between two swatches of cloth or a synthetic fabric material, which may be the outer layer 10o and inner layer 10i (see FIG. 8). In embodiments, the cloth or fabric covers the foam discussed above that allows the body 10 to flex without deforming as is discussed in further detail below.

FIG. 3B illustrates exemplary dimensions of the nose piece of the mask 300, 400 however, these dimensions may vary depending on the size of the user's head. In embodiments, the portion of the body 10 that bridges the user's nose may be elastic to provide for a better fit and for comfort. In embodiments, the one or more spacers may be a nose piece configured to sit on a user's nose when the mask 100, 200, 300, 400 is on the user's face.

In embodiments, the body 10 may include one or more speakers which may be used by a user to listen to music. In embodiments, the one or more speakers may be integrated into the flexible body 10 or may be attached thereto at a position near the user's ear when the mask 100, 200, 300, 400 is in place on the user's head. In embodiments, the speaker may be a wireless speaker that may wirelessly communicate with a music source, or other audio source, such as a smart phone, tablet, set top box, laptop computer, to name a few, and reproduce sound based on audio information provided. In embodiments, a power source may be included in the body 10 or the speaker to power the speaker. In embodiments, the power source may be a battery. In embodiments, the power source maybe a rechargeable battery and the body 10, or speaker, may include charging circuitry connected to the battery to provide for recharging. In embodiments, the speaker may be a BLUETOOTH speaker, however, any wireless communication system or protocol may be used. In embodiments, a controller may be provided in the body 10 or in the speaker to control receipt of audio information and reproduction of sound by the speaker. In embodiments, a wireless transmitter/receiver (transceiver) may be included in the speaker or elsewhere in the body 10 and connected to the speaker to receive the audio information wirelessly. In embodiments, the controller may control the transceiver and the speaker. In embodiments, the body 10 or the speaker may include an input device that may be used to turn the speaker on and off.

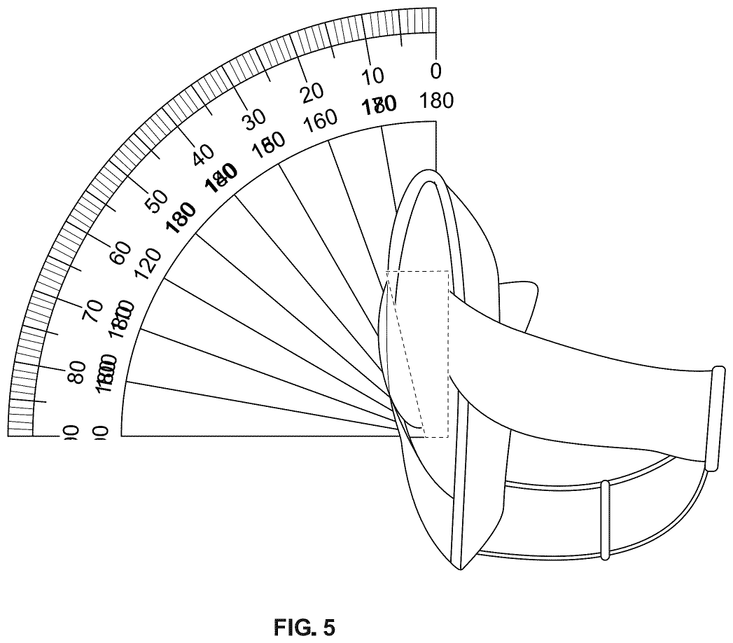

In embodiments, the body 10 may be configured to provide a desired pantoscopic tilt of the lenses 22a, 22b. In embodiments, the pantoscopic tilt is an indication of the angle between the top of the lens and the bottom of the lens. In embodiments, this angle may be between 0 and 15 degrees. In embodiments, the angle may be between 5 and 15 degrees. In embodiment, the angle may be larger. In embodiments, the body 10 may be configured to provide a desired pantoscopic tilt of the lenses 22a, 22b as generally indicated in FIG. 5, which illustrates an exemplary tilt. In embodiments, other pantoscopic tilts may be used. In embodiments, the body 10 may be configured to provide a desired vertex distance, the distance between a cornea of the user's eye and the back of the lenses 22a, 22b. In embodiments, the vertex distance may be between 9 mm and 13 mm. In embodiments, the vertex distance may be lower than 9 mm and may be larger than 13 mm. In embodiments, the flexible body 10 flexes to accommodate the user's face while positioning the lenses 22a, 22b at the desired pantoscopic tilt and vertex distance. In embodiments, the body 10 may be configured to provide a desired pupillary distance, that is the distance between the pupils. In embodiments the pupillary distance may be between 60 mm and 64 mm. In embodiments, the pupillary distance may be less than 60 mm. In embodiments, the pupillary distance may be greater than 63 mm.

In embodiments, the bezel 10c may be provided around the openings 10a, 10b as indicated in FIG. 6A. In embodiments, the bezel 10c may include a slot S (see FIG. 6B) that may be configured to receive a lens 22a or 22b. In embodiments, each lens 22a, 22b may include a peripheral seal 22c (see FIG. 7) surrounding the periphery of the lens 22a, 22b. In embodiments, the seal 22c may be a polycarbonate, silicone, or a combination of the two. In embodiments, the seal 22c may be received in the slot S. In embodiments, as noted above, the lenses 22a, 22b may be secured in place using heat treatment, ultrasonic welding, or any other suitable means. In embodiments, as noted above, the body 10 may include one or more pockets which may be aligned with the openings 10a, 10b such that the lenses 22a, 22b may be mounted in the openings via the pockets.

In embodiments, as can be seen in FIGS. 6b and 8, the slot S may be formed by an outer element 51 and an inner element S2. In embodiments, the outer element 51 and inner element S2 may be made of a self-adhering silicone and may be joined to provide the slot S. In embodiments, the slot S may be about ⅜ inch wide. In embodiments, the outer element 51 and inner element S2 may be secured to the outer layer 10o of the body which surrounds the exterior of the body. As illustrated in FIG. 8, the outer layer 10o facing forward is connected to the outer element 51 and the inner layer 10i facing the user is connected to the inner element S2. The bezel 10c may include the outer layer 10o and inner layer 10i which may define the eye opening 10a or 10b, the outer element 51 and inner element S2, and the seal 22c which holds the lens 22a, or 22b in place in the opening 10a or 10b.

In embodiments, the outer layer 10o and inner layer may be made of a soft material which may be silk or satin. In embodiments, the outer layer 10o and inner layer 10i may cover the foam portion as discussed above. In embodiments, the outer layer 10o and inner layer 10i may be any soft desired fabric or cloth. In embodiments, the outer layer 10o and inner layer 10i may be formed by one piece of cloth or fabric. In embodiments, the outer layer 10o and inner layer 10i may be one or more pieces of cloth or fabric joined together around the foam or other filler to form the flexible body 10.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A mask comprising:
a flexible body;
a strap connected to the flexible body and configured to secure the flexible body to a user's face;
at least one lens mounted in the flexible body such that the at least one lens is aligned with an eye of the user when the flexible body is secured to the user's face; and
at least one cover element movably mounted on the flexible body to move between a closed position in which the at least one cover element covers the at least one lens and an open position in which the at least one lens is uncovered such that the user sees through the at least one lens.

2. The mask of claim 1, wherein the flexible body comprises:
a first opening configured to receive the at least on lens; and
a first fastening element configured to hold the at least one cover element over the first opening to cover the at least one lens.

3. The mask of claim 1, wherein the flexible body comprises:
an outer surface made of a soft material; and
at least one foam element covered by the soft material.

4. The mask of claim 3, wherein the soft material is silk.

5. The mask of claim 3, wherein the soft material is satin.

6. The mask of claim 3, wherein the foam is memory foam.

7. The mask of claim 2, wherein the first fastening element is a hook and loop type fastener.

8. The mask of claim 2 further comprising:
a second lens mounted in the flexible body such that the second lens is aligned with a second eye of the user when the flexible body is secured to the user's face; and
a second cover element movably mounted on the flexible body to move between a closed position in which the second cover element covers the second lens and an open position in which the second lens is uncovered such that the user sees through the second lens.

9. The mask of claim 8, wherein the flexible body further comprises:
a second opening configured to receive the second lens; and
a second fastening element configured to hold the second cover element over the second opening to cover the second lens.

10. The mask of claim 1, wherein the strap is integrated into the flexible body.

11. The mask of claim 1, further comprising a securing element configured to adjustably secure the strap to the flexible body such that a length of the strap is adjustable.

12. The mask of claim 1, further comprising a securing element configured to releasably secure the strap to the flexible body.

13. The mask of claim 2, wherein the flexible body comprises a first bezel element surrounding the first opening and configured to receive the at last one lens.

14. The mask of claim 13, wherein the first bezel element includes a slot configured to receive the at least one lens.

15. The mask of claim 14, wherein the at least one lens includes a peripheral seal extending around a periphery of the at least one lens and sized to fit in the slot.

16. The mask of claim 1, wherein the at least one lens is a corrective lens.

17. The mask of claim 1, wherein the flexible body is configured such that the at least one lens is mounted in the flexible body at a desired pantoscopic tilt.

18. The mask of claim 1, wherein the desired pantoscopic tilt is between 5 degrees and 15 degrees.

19. The mask of claim 1, wherein the at least one lens is a blue light blocking lens.

20. The mask of claim 1, wherein the at least one lens is mounted in the flexible body to provide a desired vertex distance between a cornea of the user and a back of the at least one lens.

21. The mask of claim 8, wherein the body is configured to provide a desired pupillary distance between a center of the at least one lens and a center of the second lens.
